(19) United States
(12) Patent Application Publication
(10) Pub. No.: US 2026/0263257 A1
(43) Pub. Date: Sep. 10, 2026

(54) Pad having movable raised portions

(71) Applicant: BAUERFEIND AG, Zeulenroda-Triebes (DE)

(72) Inventors: Dominique PANZER, Zeulenroda-Triebes (DE); Thorsten JÄGER, Zeulenroda-Triebes (DE); Beatrix BAUERFEIND-JOHNSON, Zeulenroda-Triebes (DE)

(21) Appl. No.: 19/489,251

(22) PCT Filed: May 29, 2024
(86) PCT No.: PCT/EP2024/064740

§ 371 Date: Dec. 1, 2025

(30) Foreign Application Priority Data
Jun. 2, 2023 (DE) 10 2023 205 183.4

(51) Int. Cl. A61F 5/32

(52) U.S. Cl. CPC A61F 5/32

(57) ABSTRACT

The invention relates to pads, in particular for orthopaedic aids, comprising at least one protuberance element. The invention also relates to the use of the pads according to the invention in orthopaedic aids or sports aids, in particular orthoses or bandages, as well as orthopaedic aids or sports aids, in particular orthoses or bandages, which have pads according to the invention.

The invention relates to pads, in particular for orthopaedic aids, comprising at least one protuberance element. The invention also relates to the use of the pads according to the invention in orthopaedic aids or sports aids, in particular orthoses or bandages, as well as orthopaedic aids or sports aids, in particular orthoses or bandages, which have pads according to the invention.

Pads are known in various embodiments and for various prophylactic and therapeutic applications. Various pads are known, for example, from DE 27 22 563 C2, EP 0 598 291 A1 and EP 0 600 218 A2.

Also known from DE 297 01 001 U1 are pads of two materials or components. EP 0 496 071 A1 describes a pressure pad made of a softer material in which at least one friction core made of a hard or incompressible material is arranged. This friction core is intended to provide a friction massage, thus a massage by movement friction of pain points which is generated by the movement of the core relative to the soft tissue of the pad wearer.

DE 10 2019 212 740 B3 describes pad pressure elements that protrude from the pad in the direction of the wearer.

The pads from the prior art can flatly exert a pressure on the adjacent tissue or distribute and/or shield the incidental pressure on the face, but with these pads it is not or only insufficiently possible to exert a specific pressure, for example a stronger or weaker pressure on specific points.

Also, the pressure is applied vertically to the skin with pads from the prior art. A lateral pressure, which in particular leads to a movement of the pressure elements and thus creates a massage effect beyond the simple pressure, is not possible like this.

Also, the pressure exerted by the pressure elements of the prior art is often insufficiently strong.

The technical problem underlying the present invention is therefore the provision of improved pads, in particular such pads which can exert a specific point pressure in addition to a flatly pressure. In particular, the technical problem underlying the present invention is to provide a pad which leads to an improved massage effect, in particular to a massage effect by movement of the pressure body. In particular, the technical problem underlying the present invention is the provision of a pad which exerts a comparatively strong pressure, in particular in combination with the massage effect.

The technical problem underlying the invention is solved by providing a pad of claim 1.

The technical problem underlying the invention is solved by the provision of a pad for orthopaedic aids or a sports aid, comprising a pad base element made of a first material, wherein the pad base element has an effective face and an abutment face, wherein at least one protuberance element is assigned to the pad base element, wherein the protuberance element has at least one protuberance area which projects out of the effective face of the pad base element, characterised in that the protuberance element has an abutment area which projects out of the abutment face of the pad base element.

In the present invention, the protuberance element thus projects not only out of the effective face of the pad base element, as is realised in conventional pads, but additionally also out of the abutment face. In cross-section, there are therefore three planes of the pad: in the centre, the pad base plane, in which the pad base element lies, from this pad base plane in the direction of the person wearing the pad, the effective plane, in which the protuberance area of the protuberance element lies, and in the other direction of the pad base plane, in particular, if preferably present, in the direction of a pad support, the abutment plane, in which the abutment area of the protuberance element lies. Between the effective plane and the abutment plane therefore the pad base plane is present, which can be understood as the ‘thickness’ of the pad base body.

In this way, the at least one protuberance element can be triggered in an advantageous manner by means of the abutment area in a second plane facing away from the user's body, thus the abutment plane. This Triggering ‘from the outside’ increases the pressure exerted by the protuberance elements in an advantageous manner. The pressure from the outside, for example from the pad support, for example in the form of a knitted fabric or a base body of the bandage on these abutment areas of the protuberance elements in the abutment plane, triggers the protuberance elements and also generates a different massaging movement in the user through the protuberance areas on the effective face facing the body, thus in the active plane. The basic movement of the protuberance elements when triggered by the abutment areas is a tilting, in particular outwards, thus away from a common centre point. The tilting movement occurs in an advantageous manner when the bandage is closed and also during movement. If the applied force is reversed, however, the tilting movement of the protuberance elements can also take place inwards, thus towards their common imaginary centre.

For the function according to the invention, the counter-pressure does not necessarily have to be generated by the pad support. It is also possible for this counter-pressure to be generated, for example, by a chair back, a hand placed on it, etc., without the pad support covering the relevant area of the pad. The protuberance spring technology according to the invention via the abutment area can also reach the effect if the pad is attached directly to the body part, for example by sticking, as long as a force can additionally be applied in the abutment plane. Thus, although a pad support can be advantageous, it is only optional and not mandatorily present.

The combination of pressure application on both sides of the pad results in an advantageous manner in a complex movement pattern of the protuberance elements, which is concretely different for each movement of the patient.

In an advantageous manner, it turned out that this lateral movement of the protuberance elements also occurs chaotically depending on the direction of the force vector introduced, which enhances the massage effect.

Compared to elevations from the prior art, the protuberance elements according to the invention spring much more freely and flexibly, and thus in particular also more chaotically. This effect is advantageous, as it is more difficult to adjust for the mechano-receptors lying directly under the skin surface to the ever-changing spring movement of the protuberance elements. This leads to a prolonged effect of the massage.

Since in a manual therapy it is ‘chaotically’ massaged, as no two grips of the masseur are 100% identical, the massage effect resulting from the pad according to the invention is more similar to that of a manual massage than the massage effect resulting from conventional pads. The connection of protuberance elements with abutments leads in an advantageous manner to movable elevations which enable an intermittent massage principle. While immobile elevations on a pad only have one pressure effect that affects on the skin sensors, which, however, become accustomed to the pressure after a relatively short time and no longer emit any effective signals, the movable protuberance elements according to the invention lead to an alternating pressure, such as that for example exerted by a therapist during a massage, so that the pressure effect on the lymphatic and blood vessels remains. This can be particularly advantageous in the case of a knee pad, for example, if a corresponding massage effect is put on the meniscus base and the Hoffa's fat bodies. The protuberance elements move chaotically in different directions in an advantageous manner depending on the force introducing vectors.

In the context of the present invention, a pad is understood in particular to be a pressure pad, the shape of which is formed in particular by the pad base element. The pad base element may have any suitable shape. Conventional pads typically have flat base faces as the pad base element. Protrusions, in the present case in particular the protuberance elements, can then project out of such a base face. A pad according to the invention has, in particular, a first base face or base surface which faces the user when the pad is in use. This is the effective face. Preferably, a pad is essentially flat. Such a pad then has a second base face or base surface which faces away from the user when the pad is in use. This is the abutment face.

The typical use of a pad is known to the person skilled in the art. Pads, including the pads according to the invention, are used in particular to exert a pressure on certain parts of the body, for example in the area of the back or in the area of the kneecap. The pads are positioned and pressed onto the corresponding body area using further devices, in particular sports aids, bandages or orthoses.

Preferably, the pad base element is plate-shaped, thus a plate. The plate can be flat or curved or arched. The plate can have one or more recesses.

The protuberance area and the abutment area can be positioned next to or on top of each other on the protuberance element. For example, the protuberance area and the abutment area can be two different bodies that projects out in different directions, or they can be formed from a single body that projects out of both the effective face and the abutment face.

In a preferred embodiment, the protuberance element has a web element via which it is attached to the pad base body. The web element can be located between the abutment area and the pad base body or the abutment area forms the web element.

In an advantageous manner, the web leads to a defined, controlled deflection movement rather than a springing that is possible in all directions, such as a ‘floating springing’.

In a preferred embodiment, the at least one protuberance element is movable.

Preferably, the protuberance element is a pressure enhancing element. A pressure enhancing element serves to exert a stronger pressure at a point than the flat pressure exerted by the pad base element.

In a preferred embodiment, the pad has at least two protuberance elements. In a preferred embodiment, the pad has at least two to at most twenty protuberance elements. In a preferred embodiment, the pad has a plurality of protuberance elements.

The plurality of protuberance elements can be positioned on the pad base element of the pad in such a way that they exert the precise pressure on specific points, for example on trigger points, on acupuncture points or on infrapatellar fat bodies, when the pad is used.

In addition to the at least one protuberance element, the pad can also have friction elements or other pad pressure elements, for example made of the pad base element material.

In a preferred embodiment, the pad base element and the at least one protuberance element are made of one piece and/or of the same material. In a preferred embodiment, the pad base element and the at least one protuberance element are made of one piece. In a preferred embodiment, the pad base element and the at least one protuberance element are made of the same material.

In a preferred embodiment, the pad base element has at least one hole, wherein the at least one protuberance element is positioned in the hole.

The protuberance element has according to the invention an abutment area and a protuberance area. The protuberance element is preferably connected to the pad base element via the abutment area or to the abutment area. The abutment area lies therefore preferably between the end of the protuberance element that forms the protuberance area and the end of the protuberance element that is connected to the pad base element, or the abutment area forms the end of the protuberance element that is connected to the pad base element.

In an alternative embodiment, in particular in the case of several protuberance elements connected to each other, the protuberance area can be connected to the pad base element, thus the protuberance area lies between the pad base element and the abutment area. Preferably, in such an embodiment of several protuberance elements, the abutment areas of the protuberance elements are connected to each other.

In a preferred embodiment, the abutment region of the at least one protuberance element has a contact pressure face and/or is present in the central longitudinal area of the at least one protuberance element and/or is present t the web element of the at least one protuberance element. In a preferred embodiment, the abutment area of the at least one protuberance element has a contact pressure face. In a preferred embodiment, the abutment area of the at least one protuberance element is present in the central longitudinal region of the at least one protuberance element. In a preferred embodiment, the abutment area of the at least one protuberance element is present at the web element of the at least one protuberance element.

The contact pressure face of the abutment area can be designed in various shapes, for example anvil-shaped, wing-shaped or disc-shaped The contact pressure face can be designed with different geometries for better pressure absorption in the abutment plane. Depending on this contact pressure face geometry, a larger or smaller force can be returned to the effective plane in an advantageous manner. Larger pressure faces of the abutment area have a greater force input into the active plane and have a harder effect.

Several contact pressure faces can also be provided on an abutment area, for example two mirror symmetrical contact pressure faces.

In a preferred embodiment, the abutment area of the at least one protuberance element is configured wing-shaped or plate-shaped.

In the unpressed state, the protuberance area preferably protrudes from the pad base body, thus the pad base plane, at an angle of approximately 90°, in particular 90° in the direction of the user. However, in the unpressed state the protuberance area can also protrude from the pad base body, thus the pad base plane, at a different angle in the direction of the user.

For a higher flexibility of the at least one protuberance element, it can contain thinnings or thickenings, for example in the protuberance area, in the abutment area between the protuberance area and the abutment area or between the abutment area and the end of the protuberance element that is connected to the pad base element. Film hinges, wedge shapes and the like are also possible.

The pad has at least one protuberance element, preferably two, three, four, five, ten or more protuberance elements. Two or more, for example three, protuberance elements can be connected to each other, in particular connected to each other via webs. Preferably, two, three or more protuberance elements are connected to each other via their abutment areas.

It is therefore preferable provided that several, in particular two or three protuberance elements can be connected to each other via their abutment areas, thus in the abutment plane, in particular connected to each other via webs. This allows 2, 3, 4 or other ‘protuberance nests’ to be formed from several connected protuberance elements.

In addition to single protuberance elements, any number of protuberance elements can also be connected in the abutment plane, in particular by means of webs, for example from 2 to 10 protuberance elements, in particular from 2 to 5 protuberance elements, for example three protuberance elements or four protuberance elements. In the event of a direct central pressure impact in the abutment plane of such a protuberance nest, these several protuberance elements evade outwards. If pressure is not applied in the centre but offset, the protuberance elements on the side facing the body spring in different directions.

It can also be provided that the pad has some isolated protuberance elements and some protuberance elements connected to each other.

In a preferred embodiment, the pad base element and the at least one protuberance element are configured in one piece, in particular they are formed from the same material.

In a preferred embodiment, the pad base element is at least partially embedded in a sheath. In a preferred embodiment, the pad base element is completely embedded in a sheath.

Thus, the shape of the sheath can determine the shape of the pad.

Preferably, the pad base element is completely enclosed by the sheath, for example moulded into the sheath.

Due to the preferred complete sheathing of the at least one pad base element with a sheathing material, in particular a softer sheathing material, for example silicone, it results not only in the described application-related advantage, but also in an advantageous manner in a simpler production possibility of the pad according to the invention, since the second base face of the pad base body, which faces away from the user, thus the abutment face, is producible flat without further ado despite the abutment area of the protuberance element.

Suitable materials for a sheath are known to the person skilled in the art and can in particular be such materials that are used for conventional flexible pads consisting of one component.

Preferably, the sheath has a viscoelastic material at least in the area of the at least one protuberance. Preferably, the sheath is viscoelastic at least in a partial area. Preferably, the sheath is viscoelastic. Preferably, the sheath consists of a viscoelastic material.

Preferably, the material of the sheath is plastic, silicone or rubber. The material can also be a thermoplastic elastomer. Polyurethanes, for example, are also suitable.

Preferably, the material of the sheath is bendable and/or stretchable, so that the sheath can adapt to the shape of the user's body when applying the pad.

Preferably, the sheathing material is not a textile material. However, in an alternative embodiment, the sheath may additionally be surrounded by a textile sheath or otherwise coated.

The sheathing material can of course also consist of mixtures of materials, in particular of at least two of the materials mentioned.

Preferably, the material of the pad base element is a flexible material, in particular a flexible plastic. In a preferred embodiment, the material of the pad base element is plastic, silicone or rubber. The material of the pad base element can of course also consist of mixtures of materials, in particular of at least two of the materials mentioned. Preferably, the pad base element and the at least one protuberance element consist of the same material or are of the same material, thus are of one piece. Preferably, the material of the protuberance element is a flexible material, in particular a flexible plastic. In a preferred embodiment, the material of the protuberance element is plastic, silicone or rubber. The material of the protuberance element can of course also consist of mixtures of materials, in particular of at least two of the materials mentioned.

Thus, the protuberance element-pad base element construction according to the invention can be produced in an advantageous manner simply by means of injection moulding technology, for example.

However, the protuberance elements, in particular their protuberance areas, can also be manufactured from several components.

According to the invention, the second material of the sheath is softer than the first material of the pad base element.

In a preferred embodiment, the material of the sheath has a Shore hardness of at least 10 Shore OO and at most 50 Shore OO.

Preferably, the material of the sheath has a hardness of at most 45 Shore OO, preferably at most 40 Shore OO, particularly preferably at most 30 Shore OO. Preferably, the material of the sheath has a hardness of at most 25 Shore OO, preferably at most 20 Shore OO, particularly preferably at most 19 Shore OO. Preferably, the material of the sheath has a hardness of at least 10 Shore OO, particularly at least 14 Shore OO, particularly preferably at least 15 Shore OO.

In a preferred embodiment, the material of the pad base element has a Shore hardness of at least 10 Shore D, preferably at least 14 Shore D and at most 80 Shore D. In a preferred embodiment, the material of the pad base element has a Shore hardness of at least 20 Shore D and at most 80 Shore D.

In a preferred embodiment, the material of the at least one pad base element has a Shore hardness of at least 20 Shore D and at most 60 Shore D.

In a preferred embodiment, the material of the at least one pad base element has a hardness of at least 20 Shore D, more preferably of at least 25 Shore D.

In a preferred embodiment, the material of the at least one pad base element has a hardness of at most 50 Shore D. In a preferred embodiment, the material of the at least one pad base element has a hardness of at most 49 Shore D, more preferably of at most 45 Shore D.

Preferably, the material of the at least one pad base element has a hardness of at least 35 to at most 45 Shore D. Preferably, the material of the at least one pad base element has a hardness of approximately 40 Shore D.

In a preferred embodiment, the material of the protuberance element has a Shore hardness of at least 10 Shore D, preferably at least 14 Shore D and at most 80 Shore D. In a preferred embodiment, the material of the protuberance element has a Shore hardness of at least 20 Shore D and at most 80 Shore D.

In a preferred embodiment, the material of the at least one protuberance element has a Shore hardness of at least 20 Shore D and at most 60 Shore D.

In a preferred embodiment, the material of the at least one protuberance element has a hardness of at least 20 Shore D, more preferably of at least 25 Shore D.

In a preferred embodiment, the material of the at least one protuberance element has a hardness of at most 50 Shore D. In a preferred embodiment, the material of the at least one protuberance element has a hardness of at most 49 Shore D, more preferably of at most 45 Shore D.

Preferably, the material of the at least one protuberance element has a hardness of at least 35 to at most 45 Shore D. Preferably, the material of the at least one protuberance element has a hardness of approximately 40 Shore D.

Preferably, the material of the at least one protuberance element and the pad base element have the same hardness. However, the material of the at least one protuberance element and the pad base element can also have a different hardness, for example the at least one protuberance element can be softer than the pad base element. In this case, the pad base element can have a hardness in the region of at least 10 to at most 80 Shore D, the at least one protuberance element can have a hardness in the region of at least 10 and at most 80 Shore A and the optional sheath can have a hardness of at least 10 and at most 50 Shore OO.

In a preferred embodiment, the material of the pad base element has a Shore hardness of at least 10 Shore A, preferably at least 14 Shore A and at most 80 Shore A. In a preferred embodiment, the material of the pad base element has a Shore hardness of at least 20 Shore A and at most 80 Shore A.

In a preferred embodiment, the material of the at least one pad base element has a Shore hardness of at least 20 Shore A and at most 60 Shore A.

In a preferred embodiment, the material of the at least one pad base element has a hardness of at least 20 Shore A, more preferably of at least 25 Shore A.

In a preferred embodiment, the material of the at least one pad base element has a hardness of at most 50 Shore A. In a preferred embodiment, the material of the at least one pad base element has a hardness of at most 49 Shore A, more preferably of at most 45 Shore A.

Preferably, the material of the at least one pad base element has a hardness of at least 35 to at most 45 Shore A. Preferably, the material of the at least one pad base element has a hardness of approximately 40 Shore A.

In a preferred embodiment, the material of the sheath has a Shore hardness of at least 10 Shore 00 and at most 50 Shore OO and/or the material of the pad base element has a Shore hardness of at least 10 Shore D, preferably at least 14 Shore D and at most 80 Shore D.

In a preferred embodiment, the material of the pad base element has a Shore hardness of at least 10 Shore D and at most 80 Shore D material, wherein preferably the sheath has a Shore hardness of at least 10 Shore OO and at most 50 Shore OO. In a preferred embodiment, the material of the sheath has a Shore hardness of at least 10 Shore-OO and at most 50 Shore-OO.

In a preferred embodiment, the pad has a sheath, wherein the protuberance element is not fixedly connected to the pad base element and is preferably embedded in the sheath. The protuberance element is thus mounted and positioned in the sheath, in particular floatingly mounted, in such a way that the protuberance area projects out of the effective face of the pad base element and the abutment area projects out of the abutment face of the pad base element without the protuberance element being fixedly connected to the pad base element via a web or otherwise. The protuberance element is therefore only positioned relative to the pad base element by the sheath. Preferably, the pad base element has at least one hole, wherein the at least one protuberance element is positioned in the hole and thus projects through it. Surprisingly, it was found that a floatingly mounted protuberance element also experiences an effectiveness increase in the effective plane via a counter-pressure. The pad, in particular the pad base element, can have any shape, in particular any pad shape known to the skilled person.

For example, the pad can be ring-shaped, in particular if it is used as a patella pad. Alternatively, the shape may be a flat continuous face. Wing-shaped appendages may be provided on one or more sides of the pads.

In a preferred embodiment, the pad is a knee pad, an ankle pad, a back pad, an elbow pad, a pad for the arm area or a pad for the abdominal area. In a preferred embodiment, the pad is a knee pad or a back pad. In a preferred embodiment, the pad is a knee pad. In a preferred embodiment, the pad is a back pad.

Preferred is a pad wherein the at least one protuberance element of the pad is positioned in the pad base element such that, in the applied state of the pad, it is localised in a region of a trigger point and can exert pressure on the trigger point.

Preferred is a pad according to the invention for use in trigger point therapy, wherein preferably the at least one protuberance element of the pad is positioned in the pad base element in such a way that it presses on a trigger point when the pad is worn.

The aim of trigger point therapy is to eliminate so-called myofascial trigger points. These are locally delimiting hardenings in the skeletal muscles.

Preferred is a pad, in particular a knee pad, as shown in the figures.

The pad according to the invention is preferably not a shoe insert or foot insert.

The pad is preferably for an orthopaedic aid or a sports aid. The pad is preferably a component of an orthopaedic aid or a sports aid. The orthopaedic aid or the sports aid can be an orthosis or a bandage.

The present invention also relates to a sports aid or an orthopaedic aid, in particular an orthosis or a bandage, comprising a pad according to the invention. The present invention also relates to a sports aid comprising at least one pad according to the invention. The present invention also relates to an orthopaedic aid comprising at least one pad according to the invention. The present invention also relates to an orthosis or a bandage comprising a pad according to the invention.

Preferred is a sports aid, an orthosis or a bandage comprising at least one pad according to the invention and a pad support, in particular a knitted fabric or a woven fabric, wherein the pad support lies against the abutment face of the pad base element of the pad.

Preferably, the pad support is a knitted fabric or a woven fabric.

The pad can be attached to the pad support in a pocket, for example. Alternatively, it can also be attached directly to the pad support, for example via Velcro fasteners or buttons.

The pad can preferably be reversibly attached to the pad support and in variable position.

Preferably, the orthosis is an elastic knitted fabric orthosis or the bandage is an elastic knitted fabric bandage, in particular a knee bandage.

The present invention also relates to the use of a pad according to the invention as a pressure pad, preferably in trigger point therapy.

The present invention also relates to a method for trigger point therapy, in which a pad according to the invention is applied to a user in a first step such that the at least one protuberance element presses on a trigger point and, in a second step, the pad according to the invention is worn in this position by the user over a period of time.

Further preferred subject-matters of the invention are apparent from the subclaims and the further independent claims.

The invention is described in more detail with reference to the following figures, without the embodiments of the invention illustrated therein being to be understood as limiting.

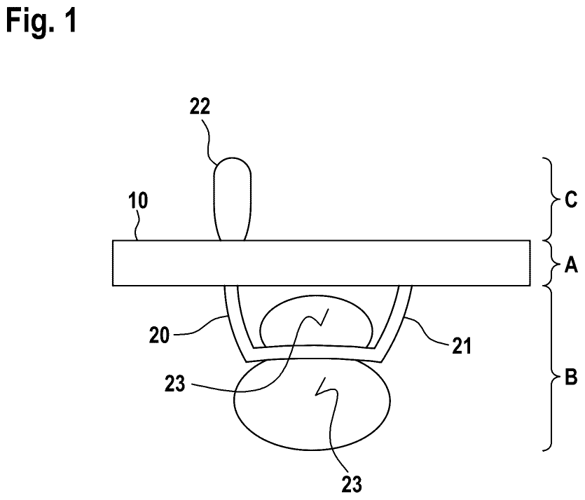

FIG. 1 shows schematically a section of a pad base element with a protuberance element in cross-section;

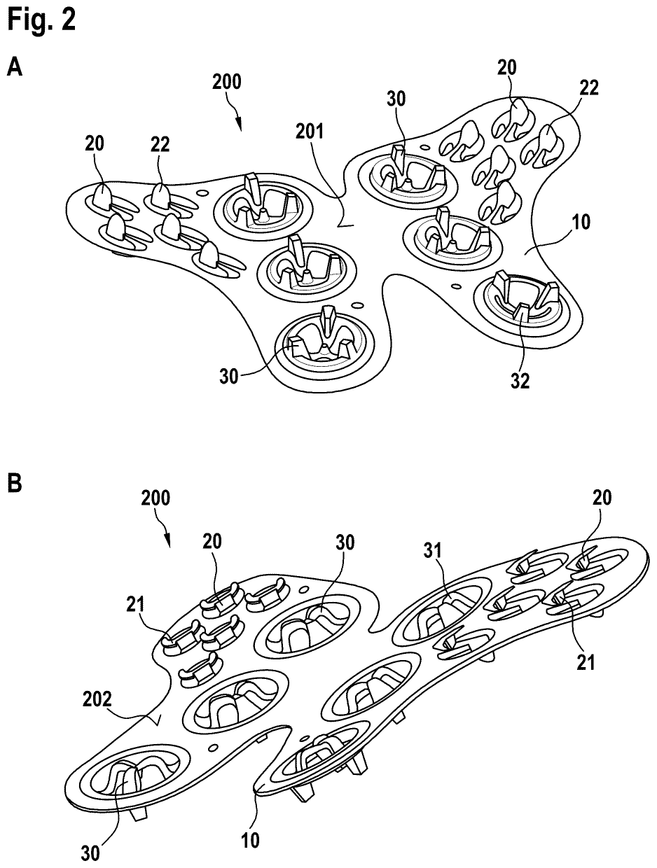

FIG. 2 shows a back pad according to the invention;

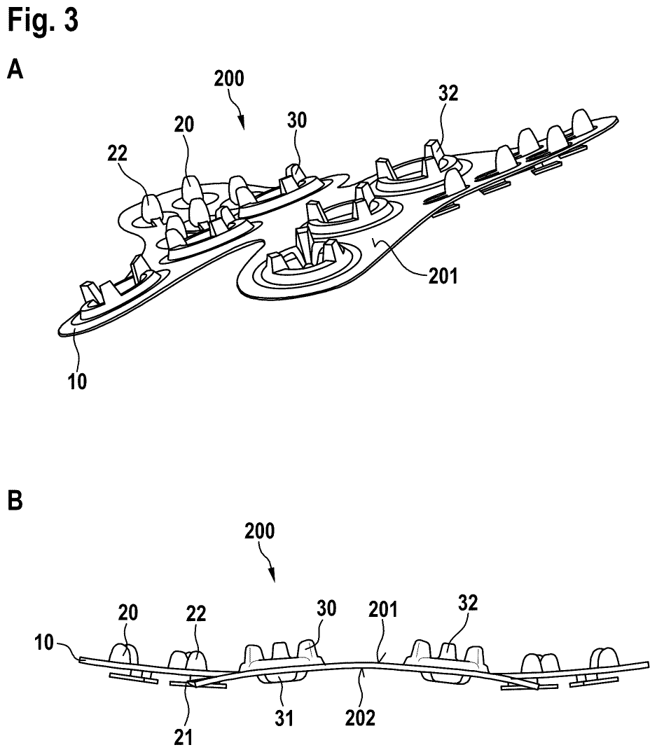

FIG. 3 shows the back pad according to the invention from FIG. 2 in two further views;

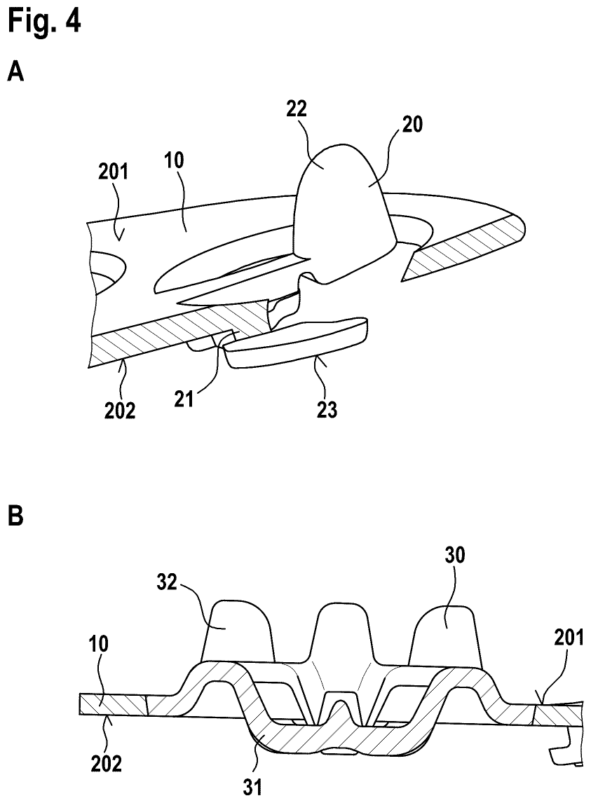

FIG. 4 shows two sections of a pad base element with a protuberance element in cross-section;

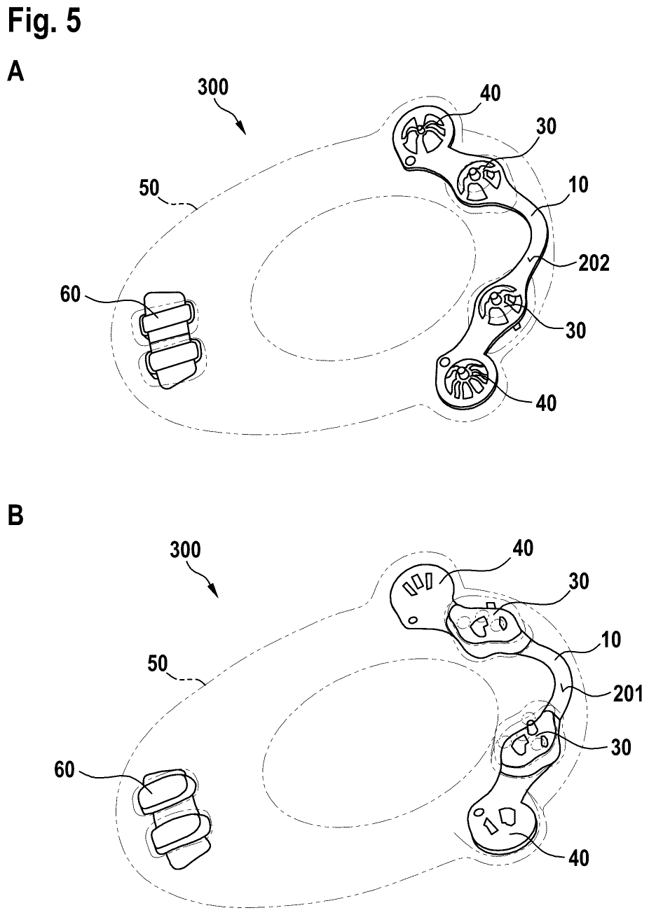

FIG. 5 shows a knee pad according to the invention;

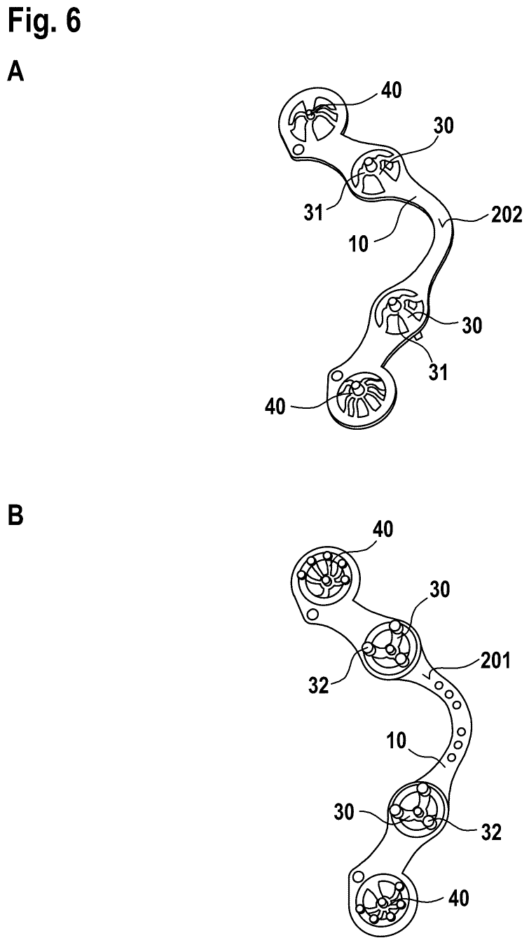

FIG. 6 shows the pad base element of the knee pad from FIG. 5;

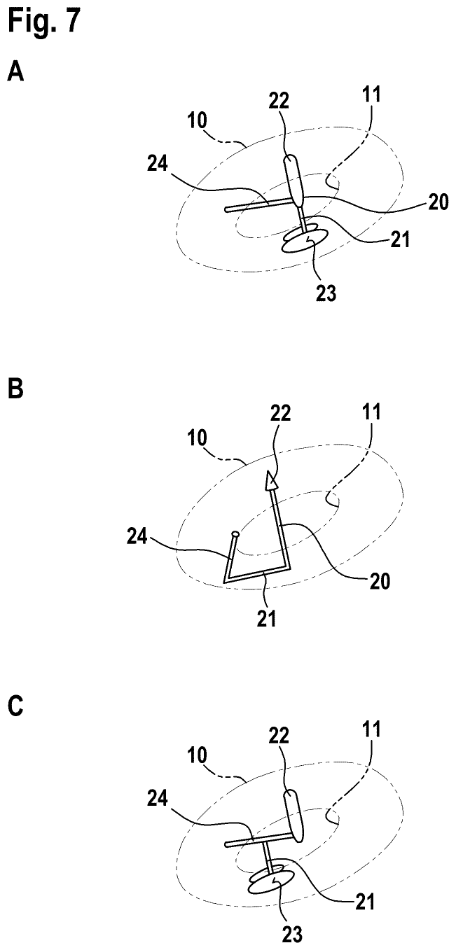

FIG. 7 shows schematically three different embodiments of a pad base element with a protuberance element;

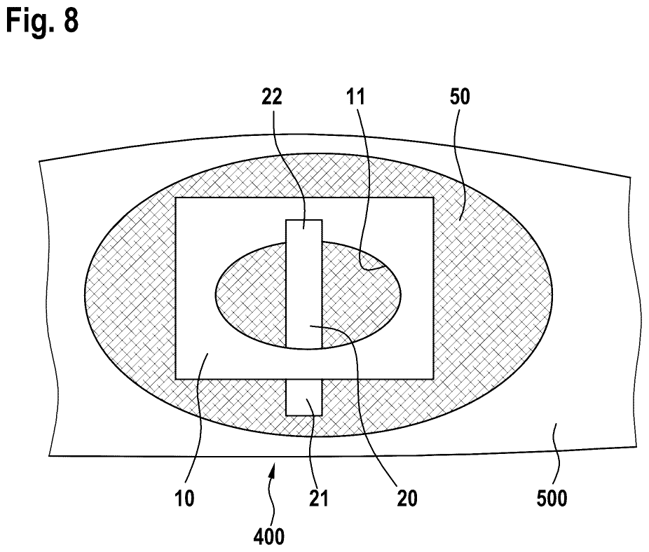

FIG. 8 shows schematically an embodiment in which the protuberance element is not fixedly connected to the pad base element

FIG. 1 shows schematically a section of a pad base element (10) with protuberance element (20) in cross-section. The pad base element (10) extends in a planar shape and, in the abstracted structure of the pad, lies in three planes in the centre plane, thus the pad base plane (A). The protuberance element (20) is connected to the pad base element (10) via the abutment area (21). The abutment area (21) lies below the pad base plane (A) and therefore in the abutment plane (B). The protuberance area (22), with which the protuberance element (20) ends, adjoins the abutment area (21). The protuberance area (22) lies above the pad base plane (A) and therefore in the effective plane (C). The pad base plane (A) therefore lies in the centre, the effective plane (C) lies from this pad base plane (A) in the direction of the person wearing the pad and the abutment plane (B) lies in the other direction of the pad base plane (A), in particular, if preferably present, in the direction of the pad support. Between the effective plane (C) and the abutment plane (B) therefore the pad base plane (A) is present, which can be understood as the ‘thickness’ of the pad base body (10).

The abutment area (21) of the protuberance element (20) has two wing-shaped, mirror symmetrical contact pressure faces (23), which can be used to increase the contact pressure.

FIG. 2 shows a back pad (200) according to the invention. In FIG. 2A the effective face (201) of the pad base element (10) facing the carrier is shown. In FIG. 2B the abutment face (202) of the pad base element (10) facing away from the carrier and towards an optional pad support is shown. The back pad has the flat pad base element (10) with several protuberance elements (20, 30) distributed over the pad base element (10). Ten protuberance elements are individual protuberance elements (20), 18 further protuberance elements are combined to form each three protuberance elements (30) connected to each other. The exact structure of the protuberance elements (20, 30) can be seen in FIG. 4.

Common to all protuberance elements (20, 30) is that they project with their protuberance area (22, 32) out of the effective face (201) of the pad base element (10) and project with their abutment area (21, 31) out of the abutment face (202) of the pad base element (10).

FIG. 3 shows the back pad (200) according to the invention from FIG. 2 in two other perspectives. FIG. 3A shows an oblique view of the effective face (201) of the pad base element (10) facing the carrier. FIG. 3B shows the side view. It can again be clearly seen how the protuberance elements (20, 30) project with their protuberance area (22, 32) out of the effective face (201) of the pad base element (10) and with their abutment area (21, 31) project out of the abutment face (202) of the pad base element (10).

FIG. 4 shows two sections of the pad base element (10) of the pad from FIGS. 2 and 3 in cross-section.

FIG. 4A shows a single protuberance element (20). The protuberance element (20) is connected to the pad base element (10) via the abutment area (21). The abutment area (21) lies below the abutment face (202). The protuberance area (22), with which the protuberance element (20) ends, adjoins the abutment area (21). The protuberance area (22) lies above the effective face (201). The abutment area (21) of the protuberance element (20) has two wing-shaped, mirror symmetrical contact pressure faces (23), which can be used to increase the contact pressure.

FIG. 4B shows three combined protuberance elements (30) connected to each other. The single protuberance elements (30) are connected to the pad base element (10) via the protuberance area (32) of each. The protuberance area (32) lies above the effective face (201). The abutment area (31) adjoins the protuberance area (32). The abutment area (31)lies below the abutment face (202). The three protuberance elements (30) are connected to each other with their respective abutment areas (31) and thus form a ‘protuberance nest’.

FIG. 5 shows a knee pad (300) according to the invention. This comprises a pad base element (10) with six protuberance elements (30), combined into two groups of three each, as well as two further optional pressure elements (40), a softer sheath (50) in the typical form of a knee pad, in which the pad base element (10) is moulded, and a further optional pressure element (60) also moulded into this. FIG. 5A shows the top view of the side of the pad (300) facing away from the body and of the pad base element (10), thus the abutment face (202). FIG. 5B shows the top view of the side of the pad (300) and the pad base element (10) facing the body, thus the effective face (201). The exact structure of the pad base element (10) and the protuberance elements (30) can be seen in FIG. 6.

FIG. 6 shows the pad base element (10) of the knee pad from FIG. 5. FIG. 6A shows the top view of the side of the pad base element (10) facing away from the body, thus the abutment face (202). FIG. 6B shows the top view of the side of the pad base element (10) facing the body, thus the effective face (201). The pad base element (10) has six protuberance elements (30), combined into two groups of three each, as well as two further optional pressure elements (40). Three single protuberance elements (30) respectively are connected to the pad base element (10) via each protuberance area (32). The protuberance area (32) lies above the effective face (201). The abutment area (31) adjoins the protuberance area (32) respectively. The abutment area (31) lies below the abutment face (202). The three protuberance elements (30) are connected to each other with their respective abutment areas (31) and thus form a ‘protuberance nest’.

FIG. 7 shows schematically three different embodiments of a pad base element (10) with protuberance element (20). In all three embodiments A, B and C, the flat shaped pad base element (10) has a hole (11) through which the protuberance element (20) extends, so that the abutment area (21) and the protuberance area (22) project out of the two faces of the pad base element (10) in opposite directions. In all three embodiments, the protuberance element (20) is connected to the pad base element (10) via a web element (24).

The three embodiments A, B and C differ in the shape and positioning of the abutment area (21), protuberance area (22) and web element (24): In embodiment A, the protuberance area (22) is peg-shaped and is located directly above the abutment area (21).

In embodiment B, the protuberance area (22) is arrow-shaped. The abutment area (21) can be regarded here as a section of the web element (24).

In embodiment C, the protuberance area (22) is peg-shaped and lies at the outer end of the web element (24). The abutment area (21) is attached to a centre area of the web element (24).

The abutment area (21) of the protuberance element (20) in embodiments A and C has two wing-shaped, mirror symmetrical contact pressure faces (23), which can be used to increase the contact pressure.

FIG. 8 shows schematically an embodiment of a pad (400) in which the protuberance element (20) is not fixedly connected to the pad base element (10). In this case, the protuberance element (20) and the pad base element (10) are moulded into a softer sheath (50). The flat-shaped pad base element (10) has a hole (11) through which the protuberance element (20) extends, so that the abutment area (21) and the protuberance area (22) project out of the two faces of the pad base element (10) in opposite directions. The protuberance element (20) is not fixedly connected to the pad base element (10) via a web or otherwise, but both are positioned by the material of the sheath (50). The floatingly mounted protuberance element (20) also experiences an effectiveness increase in the effective plane via a counter-pressure. The counter-pressure on the abutment area (21) is generated in the present case by a pad support (500), for example a knitted fabric or a woven fabric, as the pad support (500) presses on the abutment area (21) when tension is applied and this passes on the pressure through the hole (11) to the protuberance area (22) lying against the body of the support.

1. A sports aid, orthosis or bandage, comprising:
a pad for orthopaedic aids or a sports aid, the pad comprising a pad base element made of a first material, wherein the pad base element has an effective face and an abutment face, wherein at least one protuberance element is assigned to the pad base element, wherein the protuberance element has at least one protuberance area which projects out of the effective face of the pad base element, wherein the protuberance element has an abutment area which projects out of the abutment face of the pad base element, wherein the protuberance element has a web element via which it is attached to the pad base element and wherein the pad base element and the at least one protuberance element are made of one piece and/or of the same material,
and a pad support formed of a knitted fabric or a woven fabric, wherein the pad support lies against the abutment face of the pad base element of the pad.

2. The sports aid, orthosis or bandage of claim 1, wherein the protuberance element is connected to the abutment area.

3. The sports aid, orthosis or bandage of claim 1, wherein the at least one protuberance element is movable.

4. The pad of claim 1, wherein the pad base element has at least one hole, wherein the at least one protuberance element is positioned in the hole.

5. The sports aid, orthosis or bandage of claim 1, wherein the abutment area of the at least one protuberance element has a contact pressure face and/or is present in the central longitudinal area of the at least one protuberance element and/or is present at the web element of the at least one protuberance element.

6. The sports aid, orthosis or bandage of claim 1, wherein the abutment area of the at least one protuberance element is configured wing-shaped or plate-shaped.

7. The sports aid, orthosis or bandage of claim 1, wherein the pad base element is at least partially embedded in a sheath.

8. The sports aid, orthosis or bandage of claim 7, wherein the protuberance element is not fixedly connected to the pad base element and is embedded in the sheath.

9. The sports aid, orthosis or bandage of claim 1, wherein the material of the pad base element has a Shore hardness of at least 10 Shore D and at most 80 Shore D.

10. The sports aid, orthosis or bandage of claim 1, wherein the pad is a knee pad, an ankle pad, a back pad, an elbow pad, a pad for the arm area or a pad for the abdominal area.

11.-14. (canceled)

15. The sports aid, orthosis or bandage of claim 9, wherein the sheath has a Shore hardness of at least 10 Shore OO and at most 50 Shore OO.

16. A pad for orthopaedic aids or a sports aid, comprising: a pad base element made of a first material, the pad base element having an effective face and an abutment face; at least one protuberance element assigned to the pad base element, the protuberance element having at least one protuberance area that projects out of the effective face of the pad base element and an abutment area that projects out of the abutment face of the pad base element; wherein the protuberance element has a web element via which it is attached to the pad base element; and
wherein the pad base element has at least one hole and the protuberance element is positioned in the hole.

17. A sports aid, orthosis or bandage, comprising: a pad having a pad base element with an effective face and an abutment face; a plurality of protuberance elements, each having a protuberance area that projects out of the effective face and an abutment area that projects out of the abutment face, wherein at least two of the protuberance elements are connected to one another via their abutment areas to form a protuberance nest; and a pad support formed of a knitted fabric or a woven fabric that lies against the abutment face of the pad base element and is configured, in use, to apply counter-pressure to the abutment areas such that the protuberance elements tilt and generate a massage effect.

18. The sports aid, orthosis or bandage of claim 1, wherein the pad comprises a plurality of protuberance elements positioned to exert targeted pressure on trigger points, acupuncture points, or infrapatellar fat bodies.

19. The sports aid, orthosis or bandage of claim 1, wherein at least two of the protuberance elements are connected to one another via their abutment areas to form a protuberance nest comprising two to five protuberance elements.

20. The sports aid, orthosis or bandage of claim 1, wherein the pad support is an elastic knitted fabric orthosis or bandage, and the pad is reversibly attachable to the pad support in a variable position.

21. The sports aid, orthosis or bandage of claim 1, wherein at least one of the pad base element and the protuberance element comprises a flexible plastic, silicone, rubber, polyurethane, or a thermoplastic elastomer.

22. The pad of claim 12, wherein the abutment area of the protuberance element comprises two mirror-symmetrical wing-shaped contact pressure faces.

23. The pad of claim 12, wherein the protuberance element comprises at least one thinning, thickening, film hinge, or wedge-shaped portion configured to enhance a controlled deflection of the protuberance area.

24. The sports aid, orthosis or bandage of claim 13, wherein the pad is reversibly received in a pocket of the pad support or is directly fastened to the pad support by a releasable fastener comprising a hook-and-loop fastener or a button
